(19) United States
(12) Patent Application Publication
(10) Pub. No.: US 2006/0257352 A1
(43) Pub. Date: Nov. 16, 2006

(54) Mlv cosmetic product

(76) Inventor: Ingo Saar, Niederkassel (DE)

Correspondence Address: AMIN, TUROCY & CALVIN, LLP, 1900 EAST 9TH STREET, NATIONAL CITY CENTER, 24TH FLOOR,, CLEVELAND, OH, 44114, US

(21) Appl. No.: 10/569,770

(22) PCT Filed: Jun. 22, 2005
(86) PCT No.: PCT/DE05/01112

§ 371 Date: Feb. 24, 2006

(30) Foreign Application Priority Data
Jun. 28, 2004 (DE) 10 2004 031 210.9

(51) Int. Cl. A61K 8/97; A61K 8/37

(52) U.S. Cl. 424/74; 424/401

(57) ABSTRACT

The invention relates to cosmetic and/or dermatological formulations containing lecithin and essential fatty acids, which ensure a maximal skin moisture supply to the skin with a simultaneous acceleration of metabolism processes, so that the healing process of damaged skin is supported.

The invention relates to cosmetic and/or dermatological formulations containing lecithin and essential fatty acids, which ensure a maximal skin moisture supply to the skin with a simultaneous acceleration of metabolism processes, so that the healing process of damaged skin is supported. Moreover, the formulations according to invention mediate a remarkably pleasant skin feeling.

The object of the present invention is to provide cosmetic and/or dermatological formulations, which ensure a maximal skin moisture supply to the skin and accelerate the healing process of damaged skin by the acceleration of metabolism processes.

This object is solved by the technical teaching of independent claim 1. Further advantageous designs, aspects and details of the invention result from the dependent claims, the description and the examples.

Surprisingly it was found, that a cosmetic and/or dermatological formulation solves the given object, if this formulation contains: lecithin; a native oil as lipid phase with a defined preferably high content of fatty acids, especially essential fatty acids, such as sunflower oil; as well as glycerin (and/or ethylene glycol, 1,3-propanediol and/or tert-butylalcohol); propylene glycol (and/or polyethylene glycol); benzyl alcohol (and/or benzyl acetone and/or benzaldehyde); phenylethyl alcohol (and/or 2-phenylacetate); benzyl alcohol (and/or phenylethyl alcohol) and purified water.

Lecithin belongs to the phosphoglycerides group, which are also referred to as phosphatides or phospholipids. These compounds are fat-like triglycerides with two long-chain fatty acids and one phosphoric acid moiety, at which an ammonium moiety is bound as shown:

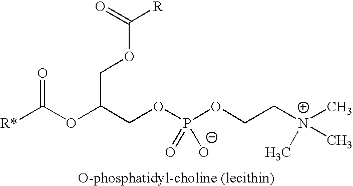

Lecithin is not a pure substance but a mixture of different fatty acid esters depending on its origin. Especially preferred is the use of lecithin from hen eggs and/or from soy. A further source is, for example, rape.

The lecithin from soy is obtained by extraction of soy with ethanol and then the lecithin is purified by chromatography and hydrated. The as-obtained phosphatidyl-choline (lecithin) contains as fatty acid moieties 80-90% by weight stearic acid, preferred about 85% by weight stearic acid and 10-20% by weight palmitic acid, preferred about 15% by weight palmitic acid. An especially suitable lecithin is Phospholipon® 80H as well as Phospholipon® 90H obtainable from PHOSPHOLIPID GmbH, Köln, Germany.

Lecithin has manifold uses in cosmetic formulations. As a component of ointments and creams it enhances the permeation of active agents into the skin and clearly improves the water retaining capability of the stratum corneum in gel formulations. Due to the increase of the skin's moisture uptake the roughness of the skin is decreased. Lecithin features as emulsifier a very low irritation potential (no washout effect). With lecithin w/o-emulsions with high water content can be prepared. Lecithin is a natural antioxidant and is used in bathing oils as emulsifier. Hydrated lecithin is suitable as a gelling and thickening agent in cosmetic formulations.

A further substantial component of the formulations according to invention are essential fatty acids, which are added to the formulation preferably in the form of suitable oils. Such suitable oils are for example sunflower oil, olive oil, corn oil, linseed oil, cod-liver oil and fish oil.

These oils of preferably native origin are added to the formulations according to invention in an amount of 3.0-20.0% by weight, preferred 6.0-16.0% by weight, further preferred 7-14% by weight, further preferred 8.0-12.0% by weight, further preferred 9-11% by weight and especially preferred 10.0% by weight.

These oils of native origin are selected according to their content as well as their composition of fatty acids, including essential fatty acids, their technological suitability and in dermatological regard. The single dermatologically active components as well as their composition are sufficiently described in the relevant technical literature. The refined sunflower oil in pharm. EUR. quality is preferred for the formulations according to invention. This refined sunflower oil (Helianthi annui oleum raffinatum) is obtained from the seed of Helianthus annuus C., has a relative density of 0.921 and a refractive index of 1.474, an maximum acid value of 0.5 (determined with 10 g of substance), a maximum peroxide value of 10, as well as the following fatty acids composition: palmitic acid 4-9% by weight, stearic acid 1-7% by weight, oleic acid 14-40% by weight and linoleic acid 48-74% by weight. A further especially suitable sunflower oil (Helianthus annuus L.) consists of 39% by weight oleic acid, 34% by weight α-linoleic acid, 13% by weight β-linoleic acid, about 4% by weight glycerol, about 9% by weight saturated fatty acids (palmitic, stearic, arachidic and linocerinic acid), carotinoids, as well as lecithin. The fatty acid fraction of the used sunflower oils contains preferably 15-35% by weight oleic acid, 50-72% by weight linoleic acid. Inter alia oleic acid is also referred to as acidum oleinicum, cis-9-octadecenoic acid, elainic acid or oleinic acid.

Essential fatty acids (EFA) are fatty acids, which can not be produced by the human body itself and hence have to be incorporated with nutrition.

Essential fatty acids always exist in the cis- and not the trans-configuration and are characterized by two or more unsaturated carbon atoms, i.e. by at least one double bond. Where the first double bond is positioned in the fatty acid chain, viewed from the methyl-end (omega-end), is characteristic. There are two types of essential fatty acids, the omega-6, derived from the cis-linolenic acid, and the omega-3, derived from the α-linolenic acid. The most important groups of omega-fatty acids are the omega-3-, -6- and -9-fatty acids.

The omega-3-fatty acids include α-linolenic acid, eicosapentaenoic acid and docosahexaenoic acid.

The omega-6-fatty acids include γ-linolenic acid, linoleic acid, arachidonic acid, delta-13-cis, 16-cis-docosadienoic acid (22:2), delta-11-cis, 14-cis-eicosa-/icosadienoic acid (20:2) and delta-7-cis, 10-cis-hexadecadienoic acid (16:2).

The omega-9-fatty acids include oleic acid (delta-9-octadecenoic acid; 18:1), gondonic acid (delta-11-cis-eicosen-/icosenoic acid; 20:1), erucic acid (delta-13-cis-docosenoic acid; 22:1) as well as nervonic acid (delta-15-cis-tetracosenoic acid; 24:1).

The highest content of linoleic acid is found in vegetable oils, such as for example safflower oil or thistle oil, sunflower oil, hemp and soy oil, and in vegetable margarine.

The triple unsaturated α-linolenic acid (9,12,15-octadecatrienoic acid) is most notably found in linseed oil (55%), walnut oil (14%), rape oil (11%) and hemp oil (7%).

The following table 1 shows important omega-3- and -6-fatty acids, their chain length and position of the double bond as well as their sources.

TABLE 1

| | Chain length and | |
|---|---|---|
| | number of double | |
| Name | bonds | Sources |
| Omega-3-fatty acids | | |
| Alpha-linolenic acid | C 18:3 | Linseed oil, soy oil, rape oil, |
| | | margarine |
| Eicosapentaenoic | C 20:5 | Sea fish (mackerel, salmon, |
| acid | | herring, sardine, tuna) |
| (=timnodonic acid) | | |
| Docosahexaenoic | C 22:6 | Sea fish |
| acid | | |
| Omega-6-fatty acids | | |
| Gamma-linolenic | C 18:3 | Hemp oil, evening primrose oil, |
| acid | | borage oil |
| Linoleic acid | C 18:2 | Thistle oil, soy oil, sunflower |
| | | oil, margarine |
| Arachidonic acid | C 20:4 | Meat, milk (only in small |
| | | amounts) |

Literature: Singer P., Was sind Omega-3-Fettsäuren, Frankfurt 1994.

For the formulations according to invention preferably unsaturated fatty acids are chosen from the group which comprises; oleic acid, eicosapentaenoic acid, timnodonic acid, docosahexaenoic acid, arachidic acid, linoleic acid, α-linolenic acid, and γ-linolenic acid, as well as mixtures of the afore-mentioned fatty acids. These mixtures especially comprise mixtures of the pure unsaturated compounds.

According to the invention oils are preferably utilized as component b, which contains at least 20%, preferred at least 25%, further preferred at least 30% and especially preferred at least 35% of omega-fatty acids. Among the omega-fatty acids especially the omega-9-fatty acids are preferred and among the omega-9-fatty acids especially oleic acid is preferred. Further the omega-6-fatty acids are especially suitable for the preparation of the formulation according to the invention and among the omega-6-fatty acids linoleic acid is preferred.

The oils used according to the invention as component b preferably contain more than 78% by weight oleic and linoleic acid, further preferred more than 86% by weight and especially preferred more than 93% by weight oleic and linoleic acid. The oils used as component b preferably contain more than 13% by weight oleic acid, further preferred more than 24% by weight oleic acid and especially preferred more than 34% by weight oleic acid. Further preferred oils as component b contain more than 38% by weight linoleic acid (α-linoleic acid+β-linoleic acid), preferred more than 47% by weight linoleic acid, further preferred more than 57% by weight linoleic acid and especially preferred more than 67% by weight linoleic acid. Another especially suitable oil is a sunflower oil (Helianthus annuus L.) containing 39% by weight oleic acid, 34% by weight α-linoleic acid and 13% by weight β-linoleic acid.

As oils are preferably utilized linseed oil, hemp oil, corn oil, walnut oil, rape oil, soy oil, sunflower oil, poppy-seed oil, safflower oil, wheat germ oil, thistle oil, grape seed oil, evening primrose oil, borage oil, black cumin oil, algae oil, fish oil, cod-liver oil and/or mixtures of the afore-mentioned oils. Especially suitable is sunflower oil. Further it is possible to add to the oils one or more essential fatty acids to provide the necessary amount of essential fatty acids.

Fish oil and cod-liver oil mainly contain eicosapentaenoic acid (EPA C20:5) and docosahexaenoic acid (DHA C22:6), in addition to a little α-linolenic acid (ALA C18:3). Omega-3-fatty acids are not only found in fish oil, but also in vegetable oils.

The following table 2 shows a listing of the fatty acid components in different oils.

TABLE 2

| | | | | Eicosa- | Docosa- |
|---|---|---|---|---|---|
| | | Linoleic | Linolenic | pentaenoic | hexaenoic |
| | Oleic acid | acid | acid | acid | acid |
| | (C 18:1) | (C 18:2) | (C 18:3) | (C 20:5) | (C 22:6) |
| Oil type | omega-9 | omega-6 | omega-3 | omega-3 | omega-3 |
| Olive oil | 70 | 10 | 0 | 0 | 0 |
| Corn oil | 30 | 60 | 1 | 0 | 0 |
| Linseed oil | 20 | 20 | 60 | 0 | 0 |
| Cod-liver oil | 25 | 2 | 1 | 12 | 8 |
| Fish oil | 15 | 2 | 1 | 18 | 12 |

The following tables 3, 4 and 5 show a listing of the monoenoic, polyunsaturated as well as acetylenic fatty acids, which are preferably used in the present invention.

TABLE 3
Monoenoic fatty acids

| Systematic name | Trivial name | Short form |
|---|---|---|
| cis-9-tetradecenoic acid | myristoleic acid | 14:1(n-5) |
| cis-9-hexadecenoic acid | palmitoleic acid | 16:1(n-7) |
| cis-6-octadecenoic acid | petroselinic acid | 18:1(n-12) |
| cis-9-octadecenoic acid | oleic acid | 18:1(n-9) |
| cis-11-octadecenoic acid | vaccenic acid | 18:1(n-7) |
| cis-9-eicosenoic acid | gadoleic acid | 20:1(n-11) |
| cis-11-eicosenoic acid | gondonic acid | 20:1(n-9) |
| cis-13-docosenoic acid | erucic acid | 22:1(n-9) |
| cis-15-tetracosenoic acid | nervonic acid | 24:1(n-9) |
| t9-octadecenoic acid | elaidinic acid | |
| t11-octadecenoic acid | t-vaccenic acid | |
| t3-hexadecenoic acid | | trans-16:1 n-13 |

TABLE 4
Polyunsaturated fatty acids

| Systematic name | Trivial name | Short form |
|---|---|---|
| 9,12-octadecadienoic acid | linoleic acid | 18:2(n-6) |
| 6,9,12-octadecatrienoic acid | γ-linoleic acid | 18:3(n-6) |
| 8,11,14-eicosatrienoic acid | dihomo-γ- | 20:3(n-6) |
| | linolenic acid | |
| 5,8,11,14-eicosatetraenoic acid | arachidonic acid | 20:4(n-6) |
| 7,10,13,16-docosatetraenoic acid | — | 22:4(n-6) |
| 4,7,10,13,16-docosapentaenoic acid | — | 22:5(n-6) |
| 9,12,15-octadecatrienoic acid | α-linolenic | 18:3(n-3) |
| | acid | |
| 6,9,12,15-octadecatetraenoic acid | stearidonic acid | 18:4(n-3) |
| 8,11,14,17-eicosatetraenoic acid | — | 20:4(n-3) |
| 5,8,11,14,17-eicosapentaenoic acid | EPA | 20:5(n-3) |
| 7,10,13,16,19-docosapentaenoic acid | DPA | 22:5(n-3) |
| 4,7,10,13,16,19-docosahexaenoic | DHA | 22:6(n-3) |
| acid | | |
| 5,8,11-eicosatrienoic acid | mead acid | 20:3(n-9) |
| 9c,11t,13t-eleostearic acid | | |
| 8t,10t,12c-calendic acid | | |
| 9c,11t,13c-catalpic acid | | |
| 4,7,9,11,13,16,19- | stellaheptaenoic | |
| | acid | |
| docosaheptadecanoic acid | | |
| | taxol acid | all-cis-5,9-18:2 |
| | pinolenic acid | all-cis-5,9,12- |
| | | 18:3 |
| | sciadonic acid | all-cis-5,11,14- |
| | | 20:3 |

Together with the essential fatty acids also non essential fatty acids or trans-fatty acids can be added to the formulation according to invention, wherein the above-mentioned percentage by weight of essential fatty acids should not fall below at least 30% by weight, preferred 40% by weight and especially at least 50% by weight.

The maximum amount of essential fatty acids should not exceed 25 g per 100 g of formulation.

TABLE 5
Acetylenic fatty acids

| Systematic name | Trivial name |
|---|---|
| 6-octadecinoic acid | tariric acid |
| t11-octadecenoic-9-in acid | santalbic or ximenynic acid |
| 9-octadecinoic acid | stearolic acid |
| 6-octadecenoic-9-in acid | 6,9-octadecenic acid |
| t10-heptadecenoic-8-in acid | pyrulic acid |
| 9-octadecenoic-12-in acid | crepenynic acid |
| t7,t11-octadecadienoic-9-in acid | heisteric acid |
| T8,t10-octadecadienoic-12-in acid | — |
| 5,8,11,14-eicosatetrainoic acid | ETYA |

The cosmetic and/or dermatological formulations according to the invention contain further glycerin as 85%-solution in water, propylene glycol respectively polyethylene glycol as consistency agent, benzyl alcohol, benzyl acetone and/or benzaldehyde as well as phenylethyl alcohol and/or 2-phenylacetate. The glycerin used can have a water content of up to 20%. Instead of glycerin a combination with glycerin ethylene glycol, 1,3-propanediol, tert-butylalcohol and/or alcohols with boiling points above 125° C., preferred above 150° C. and preferred diols or triols can be used.

Benzyl alcohol, benzyl acetone and/or benzaldehyde are added as a permeability conditioners for accelerating the metabolism processes in the skin as well as a stabilizing agents for the formulation.

Phenylethyl alcohol and 2-phenylacetate serve also as permeability conditioners for accelerating the metabolism processes in the skin and also stabilize the formulation.

Especially benzyl alcohol as well as phenylethyl alcohol lead additionally to an unexpected synergistic effect between the components of the cosmetic and/or dermatological formulation according to invention, the consequence of which is, that in the case of a very good moisture supply of the skin the metabolism processes in the skin are stimulated and accelerated and additionally a supporting of the lipid barrier takes place. Thereby, the formulations according to invention are suitable for both the treatment of damaged skin and the smoothing of crinkles in the case of longer application. Thus, formulations are provided, which are not only suitable for the dermatological application in case of for example acne, neurodermatitis and/or dermatitis but achieve simultaneously also a cosmetic effect, such as the smoothing of crinkles, by supporting the buildup as well as the regeneration of the lipid barrier and/or the stimulation as well as acceleration of the metabolism processes in the skin. Especially advantageous is the addition of benzyl alcohol and/or phenylethyl alcohol, which also contribute to a clearly increase in viscosity besides a distinct synergistic effect. This significant spontaneous increase in viscosity, especially achieved by benzyl alcohol, is substantial for the formation of the matrix or gel framework and hence for the dermatological/cosmetic function of the formulations according to invention. These two components and especially the benzyl alcohol seem to play an important role in the formation of the framework in the formulation. By the addition of these components the moisture supply of the skin is clearly increased. Another important component of the formulation is propylene glycol, which is preferably utilized in percentage amounts by weight of at least 8% by weight, further preferred 9% by weight, 10% by weight, 11% by weight, 12% by weight, 13% by weight, and especially preferred 14% by weight. The function of propylene glycol in the formation of the special framework of the formulations according to invention is not clarified yet, but an effect on the consistency of the formulation can be clearly verified.

The cosmetic and/or dermatological formulations according to invention contain lecithin in an amount of 0.5-20.0% by weight, preferred 2.0-10.0% by weight, further preferred 3.0-5.0% by weight and especially preferred 4.0% by weight.

Glycerin is utilized as a 85%-solution in water in an amount of 0.5-30.0% by weight, preferred 3.0-10.0% by weight, further preferred 5.0-7.0% by weight, further preferred 4.5-6.0% by weight and especially preferred 5.0% by weight. Ethylene glycol, 1,3-propanediol, tert-butylalcohol or other diols or triols or alcohols with boiling points above 125° C. and 150° C., respectively, are added instead of glycerin or together with glycerin in amounts, so that the afore-mentioned ranges are not exceeded or undershot.

The amount of utilized propylene glycol and/or polyethylene glycol is 0.5-50.0% by weight, preferred 6-35% by weight, 7-30% by weight, 7.5-28% by weight, 8-26% by weight, 8.5-25% by weight, 9-24% by weight, 9.5-23% by weight, further preferred 10.0-22.0% by weight, further preferred 11-21% by weight, 12-20% by weight, 13-19% by weight, further preferred 14.0-18.0% by weight, further preferred 15-17% by weight and especially preferred 16.0% by weight. Additionally it is preferred, if the afore-mentioned percentage amounts by weight represent only propylene glycol, i.e. polyethylene glycol is not present. Hence, advantageous are only small amounts of polyethylene glycol up to a maximum of 30% of the percentage amount by weight of propylene glycol, preferred up to a maximum of 20%, further preferred up to a maximum of 20%, further preferred up to a maximum of 15% and especially preferred up to a maximum of 10% of the percentage amount by weight of propylene glycol.

Benzyl alcohol, benzyl acetone and/or benzaldehyde are used in an amount of 0.1-15.0% by weight, preferred 1.0-5.0% by weight, further preferred 2.0-4.0% by weight and especially preferred 3.0% by weight.

The cosmetic and/or dermatological formulations according to invention contain phenylethyl alcohol and/or 2-phenylacetata in an amount of 0.1-15.0% by weight, preferred 0.2-5.0% by weight, further preferred 0.3-1.5% by weight and especially preferred 0.5% by weight.

The afore-mentioned components are admixed with purified water in an amount, such that the formulation is complemented to 100% by weight.

Especially preferred is a formulation of the following components: lecithin, native oil with a defined content of essential fatty acids, such as sun flower oil as well as glycerin, propylene glycol, benzyl alcohol, phenylethyl alcohol and purified water.

The compositions according to invention can be still further admixed with usual odor agents resp. perfumes, dyes, preserving agents, viscosity increasing agents and/or UV-filters. These additives serve especially for increasing the color, light and/or perfume stability of the formulations and the shelf life or imparting consistency, respectively. Suchlike additives are in total present in the formulation by not more than 10% by weight, preferred 8% by weight, further preferred 6% by weight and especially preferred 4% by weight.

The cosmetic and/or dermatological formulations according to invention can be used advantageously for the treatment of all of the forms of impure up to damaged skin. Impure up to damaged skin means for example all of the forms of acne, neurodermatitis or atypical dermatitis.

Cosmetic and/or dermatological formulations mean especially skin creams, skin lotions, milks, ointments, oils as well as balms and all of the other formulations suitable for topical application.

The following examples shall clarify the invention without limiting it.

### EXAMPLES

### Example 1

| | | Amount |
|---|---|---|
| No. | Components | (% by weight) |
| 1 | lecithin from hen's egg | 4.0 |
| 2 | oil of native origin = sun flower oil | 10.0 |
| 3 | Glycerin | 5.0 |
| 4 | propylene glycol | 16.0 |
| 5 | benzyl alcohol | 3.0 |
| 6 | phenylethyl alcohol | 0.5 |
| 7 | purified water | ad 100 |

### Example 2

| | | Amount |
|---|---|---|
| No. | Components | (% by weight) |
| 1 | lecithin from hen's egg | 4.5 |
| 2 | oil of native origin = sun flower oil | 12.0 |
| 3 | Glycerin | 4.5 |
| 4 | propylene glycol | 14.0 |
| 5 | benzyl alcohol | 2.5 |
| 6 | phenylethyl alcohol | 0.8 |
| 7 | purified water | ad 100 |

### Example 3

| | | Amount |
|---|---|---|
| No. | Components | (% by weight) |
| 1 | lecithin from soy | 3.5 |
| 2 | oil of native origin = sun flower oil | 8.0 |
| 3 | Glycerin | 6.0 |
| 4 | propylene glycol | 18.0 |
| 5 | benzyl alcohol | 3.5 |
| 6 | 2-phenylacetate | 0.6 |
| 7 | purified water | ad 100 |

### Example 4

| | | Amount |
|---|---|---|
| No. | Components | (% by weight) |
| 1 | lecithin from hen's egg | 15.0 |
| 2 | oil of native origin = sun flower oil | 15.0 |
| 3 | Glycerin | 5.0 |
| 4 | polyethylene glycol | 16.0 |
| 5 | benzyl acetone | 3.0 |
| 6 | phenylethyl alcohol | 0.5 |
| 7 | purified water | ad 100 |

### Example 5

| | | Amount |
|---|---|---|
| No. | Components | (% by weight) |
| 1 | lecithin from hen's egg | 10.5 |
| 2 | oil of native origin = sun flower oil | 18.0 |
| 3 | Glycerin | 4.5 |
| 4 | propylene glycol | 14.0 |
| 5 | phenylethyl alcohol | 3.0 |
| 6 | purified water | ad 100 |

### Example 6

| | | Amount |
|---|---|---|
| No. | Components | (% by weight) |
| 1 | lecithin from soy | 8.5 |
| 2 | oil of native origin = sun flower oil | 20.0 |
| 3 | Glycerin | 3.0 |
| 4 | tert-butylalcohol | 3.0 |
| 5 | propylene glycol | 18.0 |
| 6 | benzyl alcohol | 3.5 |
| 7 | phenylethyl alcohol | 0.6 |
| 8 | purified water | ad 100 |

### Example 7

| | | Amount |
|---|---|---|
| No. | Components | (% by weight) |
| 1 | lecithin from hen's egg | 10.5 |
| 2 | oil of native origin = sun flower oil | 18.0 |
| 3 | Glycerin | 4.5 |
| 4 | propylene glycol | 18.0 |
| 5 | polyethylene glycol | 1.5 |
| 6 | benzyl alcohol | 6.0 |
| 7 | purified water | ad 100 |

### Example 8

| | | Amount |
|---|---|---|
| No. | Components | (% by weight) |
| 1 | lecithin from hen's egg | 10.5 |
| 2 | oil of native origin = sun flower oil | 18.0 |
| 3 | ethylene glycol | 4.5 |
| 4 | propylene glycol | 22.0 |
| 5 | benzyl alcohol | 5.0 |
| 6 | benzyl acetone | 2.0 |
| 7 | purified water | ad 100 |

### Example 9

| | | Amount |
|---|---|---|
| No. | Components | (% by weight) |
| 1 | lecithin from rape | 10.5 |
| 2 | oil of native origin = sun flower oil | 18.0 |
| 3 | 1,3-propanediol | 4.5 |
| 4 | propylene glycol | 19.0 |
| 5 | benzyl alcohol | 0.8 |
| 6 | benzyl acetone | 3.6 |
| 7 | Benzaldehyde | 1.1 |
| 8 | purified water | ad 100 |

### Example 10

Proof of the Increase in Viscosity:

A suspension of 100 g of lecithin (phosphatidyl-choline 80H, Lipoid Comp.) and 900 g of water is mixed with 20 g of benzyl alcohol at 30-40° C. The viscosity before the addition of the benzyl alcohol was about 2180 mPas (Haake RV/7 (Brookfield) diving body R3, 30 rp/m, 38° C.), however after the addition of the benzyl alcohol about 54790 mPas (diving body R5, 4 rp/m, 34° C.).

The reading of the value took place after each 2 minutes.

The increase in viscosity is permanent.

1. Cosmetic and/or dermatological formulation, containing the constituents:
a) lecithin,
b) oil with high content of essential fatty acids,
c) glycerin, ethylene glycol, 1,3-propanediol and/or tert-butylalcohol,
d) propylene glycol and/or polyethylene glycol,
e) benzyl alcohol and/or phenylethyl alcohol,
f) purified water.

2. Cosmetic and/or dermatological formulation according to claim 1, containing the constituents:
a) lecithin,
b) oil with high content of essential fatty acids,
c) glycerin,
d) propylene glycol,
e) benzyl alcohol and/or phenylethyl alcohol,
f) purified water.

3. Cosmetic and/or dermatological formulation according to claim 1, further containing the constituents:
g) benzyl acetone and/or benzaldehyde,
h) 2-phenylacetate.

4. Cosmetic and/or dermatological formulation according to claim 1, wherein the lecithin is obtained from hen's egg and/or soy.

5. Cosmetic and/or dermatological formulation according to claim 1, wherein the oil of native origin has a content of essential fatty acids of at least 30% by weight, preferred 40% by weight and especially preferred of at least 50% by weight.

6. Cosmetic and/or dermatological formulation according to claim 1, wherein the essential fatty acids concern omega-fatty acids, preferred oleic acid and linoleic acid.

7. Cosmetic and/or dermatological formulation according to claim 1, wherein lecithin is contained in an amount of 0.5-20.0% by weight, preferred 2.0-10.0% by weight, further preferred 3.0-5.0% by weight and especially 4.0% by weight.

8. Cosmetic and/or dermatological formulation according to claim 1, wherein the oil of native origin is contained in an amount of 3.0-20.0% by weight, preferred 6.0-16.0% by weight, further preferred 8.0-12.0% by weight and especially 10.0% by weight.

9. Cosmetic and/or dermatological formulation according to claim 1, wherein is/are contained: glycerin as 85%-solution, ethylene glycol, 1,3-propanediol and/or tert-butylalcohol in an amount of 0.5-30.0% by weight, preferred 3.0-10.0% by weight, further preferred 5.0-7.0% by weight and especially 5.0% by weight.

10. Cosmetic and/or dermatological formulation according to claim 1, wherein propylene glycol and/or polyethylene glycol is/are contained in an amount of 0.5-50.0% by weight, preferred 10.0-22.0% by weight, further preferred 14.0-18.0% by weight and especially 16.0% by weight.

11. Cosmetic and/or dermatological formulation according to claim 1, wherein benzyl alcohol, benzyl acetone and/or benzaldehyde is/are contained in an amount of 0.1-15.0% by weight, preferred 1.0-5.0% by weight, further preferred 2.0-4.0% by weight and especially 3.0% by weight.

12. Cosmetic and/or dermatological formulation according to claim 1, wherein phenylethyl alcohol and/or 2-phenylacetate is/are contained in an amount of 0.1-15.0% by weight, preferred 0.2-5.0% by weight, further preferred 0.3-1.5% by weight and especially 0.5% by weight.

13. Use of the cosmetic and/or dermatological formulation according to claim 1 for creating a pleasant skin feeling according to skin roughness test.

14. Cosmetic and/or dermatological formulation according to claim 2, wherein lecithin is contained in an amount of 0.5-20.0% by weight, preferred 2.0-10.0% by weight, further preferred 3.0-5.0% by weight and especially 4.0% by weight.

15. Cosmetic and/or dermatological formulation according to claim 2, wherein the oil of native origin is contained in an amount of 3.0-20.0% by weight, preferred 6.0-16.0% by weight, further preferred 8.0-12.0% by weight and especially 10.0% by weight.

16. Use of the cosmetic and/or dermatological formulation according to claim 2 for creating a pleasant skin feeling according to skin roughness test.

17. Cosmetic and/or dermatological formulation according to claim 3, wherein lecithin is contained in an amount of 0.5-20.0% by weight, preferred 2.0-10.0% by weight, further preferred 3.0-5.0% by weight and especially 4.0% by weight.

18. Cosmetic and/or dermatological formulation according to claim 3, wherein the oil of native origin is contained in an amount of 3.0-20.0% by weight, preferred 6.0-16.0% by weight, further preferred 8.0-12.0% by weight and especially 10.0% by weight.

19. Use of the cosmetic and/or dermatological formulation according to claim 3 for creating a pleasant skin feeling according to skin roughness test.
